(19) United States
(12) Patent Application Publication
(10) Pub. No.: US 2006/0106421 A1
(43) Pub. Date: May 18, 2006

(54) Expansible neck bridge

(76) Inventor: Clifford Teoh, Los Altos, CA (US)

Correspondence Address: BINGHAM, MCCUTCHEN LLP, THREE EMBARCADERO CENTER, 18 FLOOR, SAN FRANCISCO, CA, 94111-4067, US

(21) Appl. No.: 10/990,163

(22) Filed: Nov. 16, 2004

(51) Int. Cl. A61B 17/08

(52) U.S. Cl. 606/213

(57) ABSTRACT

This is a medical device for bridging, and at least partially obstructing, the neck of a vascular aneurysm. In general, it is a device used to stabilize the presence of vaso-occlusive devices (such as helically wound coils) in the aneurysm. The device forms a support framework, including a base and strut(s) extending from and fixed to the base. It also includes swellable material selectively attached to the struts covering the neck portion of the aneurysm. The device, placed inside the aneurysm sac, provides therapeutic effect to the aneurysm.

### BACKGROUND OF THE INVENTION

The present invention deals with medical devices. Potentially, the devices could be used in a variety of body spaces, but the present description will be in the context of treatments for vascular aneurysms. Accordingly, one aspect of the present invention deals with a medical device for bridging, and at least partially obstructing, the neck portion of a vascular aneurysm.

Vascular aneurysms are typically formed due to a weakening in the walls of an artery. Often aneurysms are the site of internal bleeding and strokes. Left untreated aneurysms could rupture, causing potentially fatal consequences to the patient. Different medical devices have been developed for treating vascular aneurysms. Treatments commonly known as “artificial vaso-occlusion” treatments are known to be useful in treating aneurysms by filling associated undesirable vascular spaces. The space then becomes essentially closed off from the vessel and normal blood flow is restored. A variety of different vaso-occlusive devices are known to be effective for the treatment of aneurysms. The devices are introduced typically via a catheter to the site within the vasculature that is to be closed. That site may be within an aneurysm stemming from a blood vessel or within the blood vessel lumen itself.

There are a variety of materials and devices that have been used to create vaso-occlusions or emboli in the vasculature of the human body. For instance, injectable fluids such as microfibrillar collagen, various polymeric foams and beads have been used. Certain injectable fluid devices can be introduced through a catheter and are capable of forming a solid space-filling mass in a target location. Polymeric resins, particularly cyanoacrylate resins, have been used as injectable vaso-occlusive materials. Both the injectable gel and resin materials are typically mixed with a radio-opaque material to allow accurate setting of the resulted materials. Although some of these agents provide for excellent short-term occlusion, many are thought to allow vessel recanalization due to absorption of the agents into the blood. In addition, there are significant risks involved in use of cyanocrylates, and similar materials, due to the potential for misplacement. Such misplacement can create emboli in undesired areas. Generally, injectable fluid occlusion devices are somewhat difficult, if not impossible, to retrieve once they are improperly placed.

To further increase occlusive properties and thrombogenicity, a variety of vaso-occlusive devices have been treated with a variety of substances. For instance, U.S. Pat. No. 4,994,069, to Ritchart et al., describes a vaso-occlusive coil that assumes a linear helical configuration when stretched and a folded, convoluted configuration when relaxed. The stretched condition is used in placing the coil at the desired site (via passage through the catheter) and the coil assumes a relaxed configuration-which is better suited to occlude the vessel-once the device is so-placed. Ritchart et al. describes a variety of shapes. The secondary shapes of the disclosed coils include “flower” shapes and double vortices. The coils may be coated with agarose, collagen, or sugar.

As has been alluded to above, advancements in the artificial occlusion of aneurysms have occurred due to the delivery and implantation of metal coils as vaso-occlusive devices.

Vaso-occlusion coils are generally constructed of a wire, usually made of a metal or metal alloy, which is wound into a helix. Most commonly, these coils are introduced in a stretched linear form through a catheter to the selected target site, such as a particular aneurysm. The vaso-occlusion coils typically assume an irregular shape upon discharge of the device from the distal end of the catheter. The coils may undertake any of a number of random configurations used to fill an aneurysm. In some instances, vaso-occlusion coils are adapted to assume a predetermined secondary shape designed to enhance the ability to fill undesirable vascular spaces.

A variety of vaso-occlusion coils and braids are known. Tungsten, platinum, and gold threads or wires are said to be preferred. Vaso-occlusion coils have a variety of benefits including that they are relatively permanent, they may be easily imaged radiographically, they may be located at a well defined vessel site, and they can be retrieved.

In some instances, particularized features of coil designs, such as specialized mechanisms for delivering vaso-occlusion coils through delivery catheters and implanting them in a desired occlusion site, have been described. Examples of categories of vaso-occlusion coils having specialized delivery mechanisms include pushable coils, mechanically detachable coils, and electrolytically detachable coils.

Pushable coils are commonly provided in a cartridge and are pushed or plunged from an engaged delivery catheter into an aneurysm. A pusher wire advances the pushable coils through and out of the delivery catheter into the site for occlusion.

Mechanically detachable vaso-occlusive devices are typically integrated with a pusher wire and are mechanically detached from the distal end of that pusher wire after exiting a delivery catheter.

A variety of mechanically detachable devices are also known. For instance, U.S. Pat. No. 5,234,437, to Sepetka, shows a method of unscrewing a helically wound coil from a pusher having an interlocking surface. U.S. Pat. No. 5,250,071, to Palermo, shows an embolic coil assembly using interlocking clasps that are mounted both on the pusher and on the embolic coil. U.S. Pat. No. 5,261,195, to Twyford et al., shows a pusher-vaso-occlusive coil assembly having an affixed, proximately extending wire carrying a ball on its proximal end and a pusher having a similar end. The two ends are interlocked and disengaged when expelled from the distal tip of the catheter. U.S. Pat. No. 5,312,415, to Palermo, also shows a method for discharging numerous coils from a single pusher by use of a guidewire which has a section capable of interconnecting with the interior of the helically wound coil. U.S. Pat. No. 5,350,297, to Palermo et al., shows a pusher having a throat at its distal end and a pusher through its axis. The pusher sheath will hold onto the end of an embolic coil and will then be released upon pushing the axially placed pusher wire against the member found on the proximal end of the vaso-occlusive coil.

Within electrolytically detachable vaso-occlusive devices, the vaso-occlusive portion of the assembly is attached to a pusher wire via a small electrolytically severable joint. The electrolytically severable joint is severed by the placement of an appropriate voltage on the core wire. The joint erodes in preference either to the vaso-occlusive device itself or to the pusher wire. In accordance with principles of competitive erosion, parts of the wire that are not intended to erode are often simply insulated to prevent such an electrolytic response caused by the imposition of the electrical current.

U.S. Pat. Nos. 5,354,295 and its parent 5,122,136, both to Guglielmi et al., describe an electrolytically detachable embolic device. That is to say that a joint between the pusher wire and the vaso-occlusive portion dissolves or erodes when an electrical current is applied to the pusher wire.

Some vaso-occlusive devices include specialized mechanical features and/or shapes. Various shaped coils have been described. For example, U.S. Pat. No. 5,624,461, to Mariant, describes a three-dimensional in-filling vaso-occlusive coil. U.S. Pat. No. 5,639,277, to Mariant et al., describes embolic coils having twisted helical shapes and U.S. Pat. No. 5,649,949, to Wallace et al., describes variable cross-section conical vaso-occlusive coils. A random shape is described, as well. U.S. Pat. No. 5,648,082, to Sung et al., describes methods for treating arrhythmia using coils which assume random configurations upon deployment from a catheter. U.S. Pat. No. 5,537,338 describes a multi-element intravascular occlusion device in which shaped coils may be employed. Spherical shaped occlusive devices are described in U.S. Pat. No. 5,645,558 to Horton. Horton describes how one or more strands can be wound to form a substantially hollow spherical or ovoid shape when deployed in a vessel. U.S. Pat. Nos. 5,690,666 and 5,718,711, by Berenstein et al., show a very flexible vaso-occlusive coil having little or no shape after introduction into the vascular space.

One type of aneurysm commonly known as a “wide-neck aneurysm” is known to present particular difficulty in the placement and retention of vaso-occlusive devices. Furthermore, vaso-occlusive devices, in particular, vaso-occlusion coils, lacking substantial secondary shape strength may be difficult to maintain in position within an aneurysm no matter how skillfully they are placed.

Vaso-occlusive devices are typically placed in an aneurysm in the following fashion. A micro-catheter is initially steered into or adjacent the entrance of an aneurysm, typically aided by the use of a steerable guide wire. Then either the guide wire is then withdrawn from the micro-catheter and replaced by the vaso-occlusive device or a separate catheter is delivered containing the vaso-occlusive device. The vaso-occlusive device is advanced through and out of a micro-catheter, desirably being completely delivered into the aneurysm. After, or perhaps, during, delivery of the device into the aneurysm, there is a risk that the device or a portion of the device might migrate out of the aneurysm entrance zone and into the feeding vessel. The presence of the device in the feeding vessel may cause the undesirable response of an occlusion in the feeding vessel. Also, there is a quantifiable risk that blood flow in the feeding vessel and the aneurysm may induce movement of the device further out of the aneurysm, resulting in a more developed embolus in the patent vessel.

The utilization of a stent containing an outer layer of hydrogel has been presented in the art. As described, the stent can be placed in the parent vessel near the site of the aneurysm and allowed to hydrate. While potentially closing off the aneurysm neck this device unfortunately also unnecessarily seals off other parts of the vessel it contacts. This presents risks of creating stenosis or introducing emboli within the vessel. There exists a need for a device to bridge and seal the neck of the aneurysm thereby retaining the vaso-occlusive device within the aneurysm sac and allowing normal blood flow through the vessel.

Another disclosed invention involves the use of hydrogel embolizing elements disposed concentrically on a filament and deployed within the aneurysm sac. Upon hydration the vaso-occlusive device expands to fill the space within the sac. Potentially the expansion of the device could extend out from the aneurysm and disrupt normal blood flow or create emboli. A need exists for a device to bridge the neck of the aneurysm thereby retaining the vaso-occlusive device within the aneurysm sac.

As described, aneurysms present particularly acute medical risk due to the dangers associated with an inherently thin vascular wall. The utilization of vaso-occlusive devices to occlude an aneurysm without occluding the adjacent vasculature pose a special challenge. It is especially difficult when addressing wide neck aneurysms due to morphology, shape or herniation. There exists a need for a device to bridge and obstruct the wide necked aneurysms from within the aneurysm. None of the vaso-occlusive devices described above solve this problem.

### SUMMARY OF THE INVENTION

One aspect of the present invention pertains to a medical device for bridging, and at least partially obstructing, a neck portion of a vascular aneurysm. The medical device could be implantable and includes an apparatus having a base and at least one strut or lateral protrusion fixedly attached to the base. The apparatus, including the base and strut(s), is of a size and overall flexibility to lodge inside the aneurysm at the neck portion. A swellable material, such as hydrogel, is selectively applied to the strut(s) providing a therapeutic effect on the vascular aneurysm.

### BRIEF DESCRIPTION OF THE DRAWINGS

FIG. 1 is a side view of the retainer assembly.

FIG. 2 is a side view of the retainer assembly with attached marker coils.

FIG. 3 is a side view of an aneurysm emanating from the wall of a blood vessel.

FIG. 4 is a partial sectioned view of the retainer assembly being inserted within the aneurysm sac.

FIG. 5 is a side view of the retainer assembly lodged against the side wall of the aneurysm showing a coil having a swellable material attached in the vicinity of the aneurysm neck.

FIG. 6 is a partial sectioned view of the retainer assembly as the vaso-occlusive catheter delivery device is positioned.

FIG. 7 is a side view of the retainer assembly.

FIG. 8 is a side view of the retainer assembly and illustrates the procedural elements associated with using the retainer assembly.

FIG. 9 is a partial sectioned view of the aneurysm with both the retainer and vaso-occlusive devices inside the aneurysm.

FIG. 10 is a perspective view of the aneurysm closed off.

### DETAILED DESCRIPTION OF THE INVENTION

This invention involves a device and procedure for solving the problem of material flow into and out of aneurysms. A retainer assembly is placed at the mouth of the aneurysm sac, forming a barrier for blood flow into the sac or migration of other devices, such as vaso-occlusive implants, out of the sac. Vaso-occlusive implants may be placed inside the sac for filling the unwanted space. The retainer assembly includes a swellable material placed on the spanning support framework to further close off the the aneurysm sac. The swellable material absorbs fluid upon contact with blood and expands in volume. The retainer assembly with the expanded swellable material seals off the neck of the aneurysm, preventing vaso-occlusive devices from migrating into the parent vessel. The following detailed description should be read with reference to the drawings in which like elements in different figures are numbered identically.

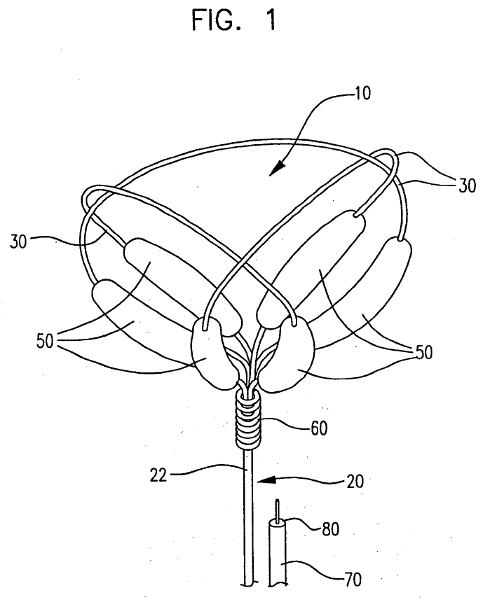

FIG. 1 illustrates a retainer assembly 10 with a base 20 and at least one strut 30 extending from the base 20 laterally. The base 20 is preferably constructed of a coil 22 with a proximal and distal end and a lumen throughout. The coil 22 is preferably made from stainless steel, or other biocompatible material including shape memory polymers, and is approximately 0.017 inches in diameter and approximately 1.0 millimeter in length. The base 20 is centrally located when a plurality of struts 30 extend outwardly in different directions from the base 20, as shown in FIG. 1. The base 20 is connected to a delivery device at the proximal end of the coil 22.

Retainer assembly 10 includes lateral struts 30 that could either be round or flat wire, or any other construction that forms a scaffolding or bridge across the neck of the aneurysm. The diameter of the wires or ribbons making up the strut elements 30 is preferably smaller than about 0.010 inches. The struts 30 may be produced from a number of materials such as metals, alloys, polymers, or other biocompatible medical device materials. Due to their desired expansion properties, superelastic alloys are commonly and desirably utilized in the strut 30 construction. Superelastic alloys are well known in the art. Nitinol, which comprises the elements nickel and titanium, is one of the more well known super-elastic alloys. It is readily commercially available and undergoes the austenitic-martensitic-austenitic transformation in a variety of temperatures between −20C and +30C.

Although it is desirable that the struts 30 of the current invention be made from superelastic alloys, other metals may in certain circumstances be appropriate. Such metals include a number of stainless steel materials and other highly elastic, if not superelastic alloys. Furthermore, it is within the scope of this invention that the struts 30 be formed from polymeric materials. Such materials as polyethylene, polypropylene, various nylons, shape memory polymers, or polytetrafluoroethylene could be chosen along with a variety of other suitable polymeric materials which are biocompatible and fit the criteria necessary for this invention.

The retainer assembly 10 has an overall shape which approximates that of the aneurysm neck in which it is placed. Specifically, the retainer assembly 10 has a number of struts 30 which extend from the base 20, loop around and rejoin themselves to the base 20 forming a spanning support frame. The lateral struts 30 are connected to the distal end of the base by methods known to those skilled in the art, such as welding, crimping, adhesive bonding, or fusing. While the preferred embodiment, as shown in FIG. 1, includes struts 30 that loop around and connect to the base 20, other types of lateral protrusions should be considered within the scope of the present invention. FIG. 1 illustratively includes three struts 30, more or fewer could be utilized and are within the scope of the invention.

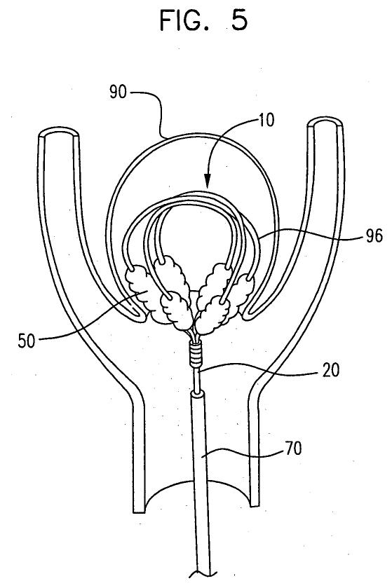

As shown, the retainer assembly 10 including three struts 30 creates a spanning “flower” shape. Although the illustrated struts 30 are generally shown to be approximately the same size and shape with respect to each other, this is not necessary. It is within the scope of this invention that the retainer assembly 10 be irregular in shape to fit within an irregularly shaped aneurysm. The length of the struts 30 is generally appropriate to form a spanning element which contacts points along the inside of the aneurismal wall (further described in FIG. 5) and connects to the base 20. While a certain minimal contact with the aneurysm wall is necessary to support the bridging struts 30 it is also possible for the retainer device to include struts 30 which extend further along the inside wall of the aneurismal sac.

As shown in FIG. 1, the struts 30 further contain an expansible material 50, such as hydrogel, selectively attached for further occluding the aneurysm neck. The material 50 is disposed on the struts 30 in locations, as shown in FIG. 1, which span and provide framework across the opening of the aneurysm sac. Upon hydration with time, the hydrogel 50 swells and effectively seals off the aneurysm by reducing the effective open area of the aneurysm neck.

The absorbent material 50 is preferably formed of a polymeric colloidal system which is biocompatible. It may be a hydrogel or other such material with properties allowing it to swell and stabilize upon exposure to body fluids. Examples of hydrogels include gels formed from homopolymers, copolymers, and/or network polymers containing: polyethylene glycol, polypropylene glycol, polyvinyl alcohol, polyvinylpyrrolidone, polyacrylates, polymethacrylates, polyacrylamides, polyethyloxazoline, polysaccharides, mucopolysaccharides, polyaminoacids, carboxy alkyl celluloses, partially oxidized cellulose, hyaluronic acid, dextran, heparin sulfate, chondroitin sulfate, heparin, agar, starch, alginate, fibronectin, gelatin, collagen, fibrin, pectins, albumin, ovalbumin, polyesters of .alpha.-hydroxy acids including polyglycolic acid, poly-DL-lactic, poly-L-lactic acid, polylactones, polyanhydrides, polyorthoesters, polydioxanone, polycaprolactones, poly(delta-valerolactone), poly(gamma-butyrolactone), and combinations thereof. The gel may further comprises a chemical cross-linking agent having two or more reactive groups in order to form chemical bridges between two or more polymeric molecules, examples of cross-linking agents include diacrylates, oligoacrylates, dimethacrylates, oligomethacrylates, divinyl ethers, certain cations, and combinations thereof.

The swellable material 50 can preferably be coated on the struts 30 or array members of the retainer assembly 10 at the neck bridging locations. Alternatively, the hydrogel 50 may be fixed to the struts 30 by a suitable biocompatible adhesive or other mechanical or chemical adhesion processes known in the art. These include spray or dip coating, chemical or plasma deposition or any other form of simple mechanical attachment or restriction. The preferred method will allow the hydrogel 50 material to remain permanently attached to the struts 30 before, during and after delivery and hydration. The bonding method will allow expansion of the assembly 10 to its secondary shape without detrimental effects to the outer layer of hydrogel 50. The hydrogel 50 may be of a type that dissolves over time and may include growth factors or other biologic materials such as collagen or fibrin to facilitate thrombosis and cell infiltration. Bioactive materials may be incorporated into the hydrogel 50, including cytokines, growth factors, extracellular matrix molecules, proteins, enzymes, enzyme inhibitors, receptors, cells, polynucleic acids, lipid-based molecules, polysaccharides, drugs and fragments or combinations of these materials. The hydrogel 50 may also incorporate radiopaque materials.

The distribution and arrangement of the hydrogel matrix 50 on the array struts 30 covering the aneurysm neck may be controllable and shaped to allow certain sized objects, such as blood, to escape while trapping embolus or PVA particles if required. The hydrogel 50 can also be used to effectively close off the neck area. A braid like structure or other pre-biased geometry, formed of hydrogel 50, can be disposed on the array struts 30 and configured to expand outward from the struts to develop a sheet, or flat panel, as opposed to possibly expanding into the parent vessel and potentially occluding blood flow. Preferential expansion may also be accomplished by the use of coatings or elements within the hydrogel 50 which restrict expansion. Changes in pH or temperature, or other external activation methods, may be used to initiate the selectively expandable hydrogel 50 in a certain direction or to a certain volume.

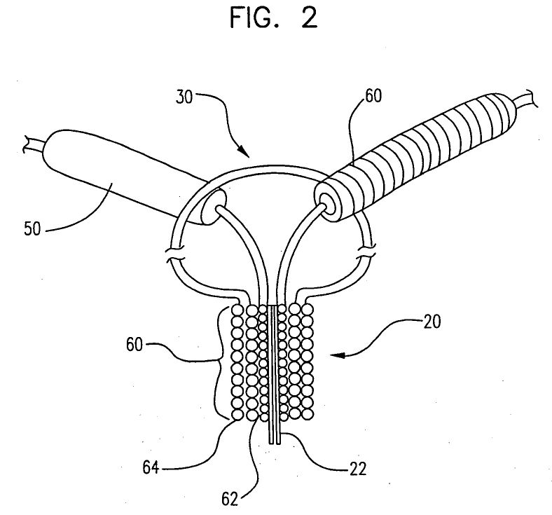

Marker coils 60 are constructed of radio-opaque material (ie. platinum) and assist in the guidance of the retainer assembly through a tubular delivery device and through a vascular system using radiography or fluoroscopy. In particular, the marker coils 60 facilitate positioning of the bridge subassembly 10 within the aneurysm. The marker coils 60 may be placed at the site of overlapping struts 30, the base of the retainer assembly 20 or any location along the strut 30 protrusions that assists in the placement of the assembly within the aneurysm. One embodiment, illustrated in FIG. 2, includes a base 20 with a marker 60 attached to the coil 22. The marker 60 includes an inner 62 and outer marker coil 64. The ends of the struts 30 are illustratively fixedly secured between the inner marker coil 62 and outer marker coil 64. The struts or lateral protrusions 30 may include marker coils 60, shown in FIG. 2 attached at the location where the struts 30 overlap. The marker coils 60 may be added to the various elements of the device by methods known within the art such as plating, crimping or wrapping the element in a radio-opaque wire or ribbon.

In accordance with one embodiment of the invention, shown in FIG. 1, the proximal portion of the base 20 is detachably connected to a severable joint 80 within a catheter 70. The severing action of the joint 80 enables the retainer assembly 10 to remain in a portion of the aneurysm 90 after the catheter 70 has been removed from the vasculature 92. There are several means for actuating the severable joint 80 but for the purpose of simplifying the description, it will be assumed that the severable joint 80 is an electrolytic severable joint 80.

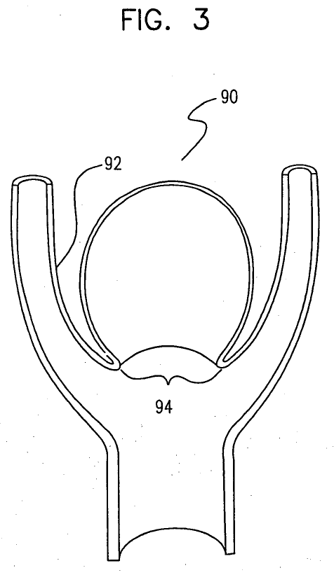

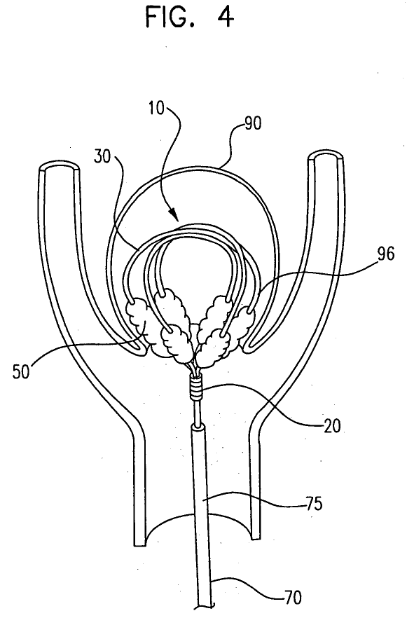

FIG. 3 illustrates a cross sectioned view of an aneurysm 90 emanating from the wall 92 of a blood vessel. A catheter 70 is shown in FIG. 4 delivering a retainer assembly 10. The retainer assembly 10 is designed to fit inside a catheter 70 for delivery through the vasculature 92. As the retainer assembly 10 is contained in the catheter 70 and delivered through the vascular 92 system it has a retracted or low profile shape. The retainer assembly 10 is positioned at the neck 94, or opening of the aneurysm, as shown in FIG. 5. The retainer assembly 10 takes its secondary, or expanded, shape as it is pushed out of the distal end of the catheter 70.

The catheter 70, as shown in FIGS. 1 and 4, contains a radio-opaque band or coil 60 at its distal end. As is known in the art, radio-opaque band 60 assists in the guidance of catheter 70 through the vasculature 92 using fluoroscopy or radiology. As shown, the distal end of catheter 70 can be guided through the neck portion of the aneurysm 94. Once the catheter 70 is inside the aneurysm 90 the retainer assembly 10 can be pushed out of the distal end of the catheter 70 thus providing a device for aneurismal occlusion, as seen in FIG. 5.

In order for the retainer assembly 10 to be implanted in the aneurysm 90, the base 20 must be detachably connected to a catheter 70, see FIG. 5. As stated, one method for detachment of the base 20 from the catheter 70 is by activation of an electrolytically severable joint 80. The severable joint 80 connects the distal end of a core wire 75, as shown in FIG. 4, to the base 20 of the retainer assembly 10. The severable joint 80 and the action of releasing the retainer assembly 10, has been described in detail in U.S. Pat. No. 6,569,179, incorporated herein for reference. There are, of course, other means for detaching the retainer assembly 10 which should be considered within the scope of the invention.

Retainer assembly 10 is further depicted in FIG. 5 showing placement against the side wall 96. Lateral struts 30 in combination with base section 20 make up the bridge retainer assembly 10. While lateral struts or protrusions 30 are illustratively wire loops, other types of struts 30 should be considered within the scope of the present invention. The struts 30 further contain an absorbent material 50, such as hydrogel, selectively attached for further occluding the aneurysm neck 94. Upon hydration with time, the hydrogel 50 swells and effectively seals off the aneurysm neck 94.

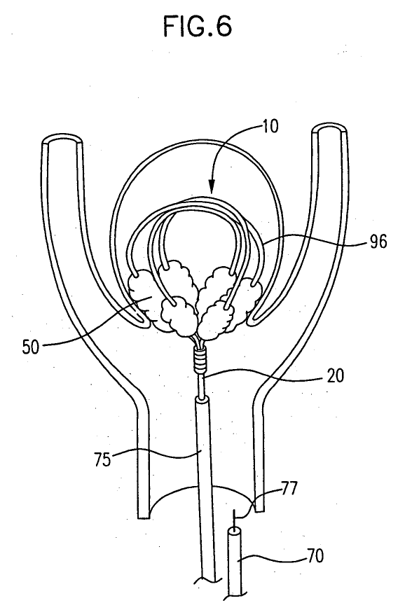

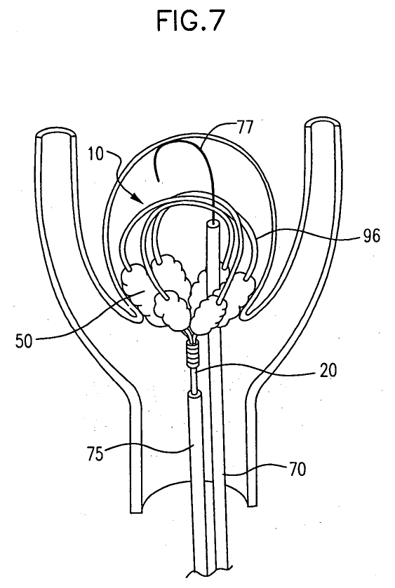

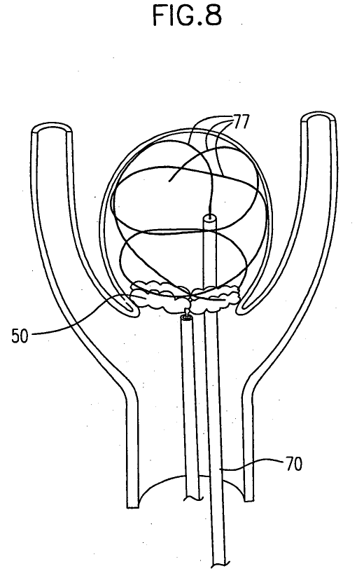

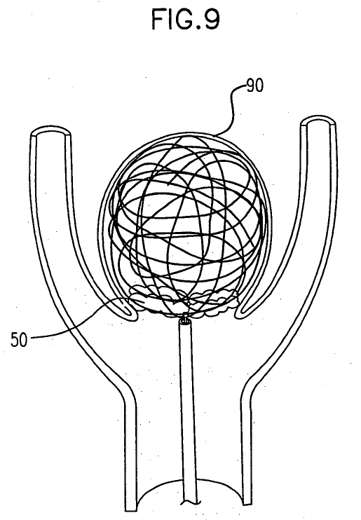

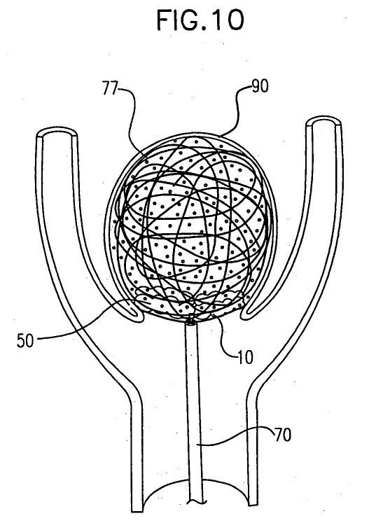

With the retainer assembly 10 in place against the inside of the aneurysm wall 94, FIGS. 6 and 7 show vaso-occlusive devices 77, such as vaso-occlusion coils, pushed through a catheter 70 and retainer assembly 10 inside the aneurysm 90. Coiling can be performed during expansion of the hydrogel 50 to stabilize the device within the aneurysm 90. FIGS. 8, 9 and 10 depict the aneurysm 90 filling with vaso-occlusive devices 77 while being retained by the bridge retainer assembly 10.

Those skilled in the art will recognize that the present invention may be altered and modified in a variety of forms other than the specific embodiments described and contemplated herein. Accordingly, it will be recognized that a variety of changes and modifications can be made without departing from the scope and spirit of the invention as described in the following claims.

What is claimed is:

1. An apparatus for bridging a neck of an aneurysm, comprising:
a base section;
at least one strut extending outwardly from the base section;
wherein the at least one strut is configured to be lodged inside an aneurysm substantially bridging the neck of the aneurysm; and
a swellable material selectively disposed on the at least one strut.

2. The apparatus of claim 1, wherein said base section is made of a super-elastic alloy.

3. The apparatus of claim 1, wherein said base section is made of stainless steel.

4. The apparatus of claim 1, wherein said base section is made of shape memory polymer.

5. The apparatus of claim 1, further comprising a radio-opaque marker attached to the base section.

6. The apparatus of claim 1, further comprising a detachable joint connected to the base section.

7. The apparatus of claim 1, wherein said at least one strut reconnects to said base section forming a loop.

8. The apparatus of claim 1, wherein said at least one strut is made of a super-elastic alloy.

9. The apparatus of claim 1, wherein said at least one strut is made of stainless steel.

10. The apparatus of claim 1, wherein said at least one strut is made of shape memory polymer.

11. The apparatus of claim 1, further comprising a radio-opaque marker attached to at least one strut.

12. The apparatus of claim 9, wherein said radio-opaque marker is made of platinum.

13. The apparatus of claim 1, wherein said swellable material is a hydrogel.

14. The apparatus of claim 11, wherein said swellable material is selected from the group comprising collagen, gelatin, fibrin, polyethylene oxide, polyacrylic acid, polyacrylamide, poly(sodium-4-styrenesulfonate), poly(3-hydroxybutyric acid), polyvinylpyrrolidone and 2-hydroxyethyl methacrylate.

15. The apparatus of claim 11, wherein said swellable material further comprises a bioactive material.

16. The apparatus of claim 15, wherein said bioactive material is selected from the group comprising cytokines, growth factors, extracellular matrix molecules, proteins, enzymes, enzyme inhibitors, receptors, cells, polynucleic acids, lipid-based molecules, polysaccharides and drugs.

17. The apparatus of claim 1, wherein the swellable material is disposed on the at least one strut at the neck of an aneurysm.

18. The apparatus of claim 1, wherein said swellable material further comprises a geometry for directing expansion.

19. An apparatus for bridging a neck of an aneurysm, comprising:
a base section;
a plurality of struts extending from the base section forming a plurality of loops;
wherein the plurality of struts are configured to be lodged inside an aneurysm substantially bridging the neck of an aneurysm; and
a swellable material selectively disposed on the plurality of struts at the neck of the aneurysm.

20. The apparatus of claim 19, wherein said base section is made of a super-elastic alloy.

21. The apparatus of claim 19, wherein said base section is made of stainless steel.

22. The apparatus of claim 19, wherein said base section is made of shape memory polymer.

23. The apparatus of claim 19, further comprising a detachable joint connected to said base section.

24. The apparatus of claim 23, wherein the detachable joint can be electrolytically activated.

25. The apparatus of claim 19, further comprising a radio-opaque marker attached to the base section.

26. The apparatus of claim 25, wherein said radio-opaque marker is made of platinum.

27. The apparatus of claim 19, wherein the plurality of struts is three struts.

28. The apparatus of claim 19, wherein said plurality of struts is made of a super-elastic alloy.

29. The apparatus of claim 19, wherein said plurality of struts is made of stainless steel.

30. The apparatus of claim 19, wherein said plurality of struts is made of shape memory polymer.

31. The apparatus of claim 19, further comprising a radio-opaque marker attached to the plurality of struts.

32. The apparatus of claim 31, wherein said radio-opaque marker is made of platinum.

33. The apparatus of claim 19, wherein said swellable material is a hydrogel.

34. The apparatus of claim 33, wherein said swellable material is selected from the group comprising collagen, gelatin, fibrin, polyethylene oxide, polyacrylic acid, polyacrylamide, poly(sodium-4-styrenesulfonate), poly(3-hydroxybutyric acid), polyvinylpyrrolidone and 2-hydroxyethyl methacrylate.

35. The apparatus of claim 33, wherein said swellable material further comprises a bioactive material.

36. The apparatus of claim 35, wherein said bioactive material is selected from the group comprising cytokines, growth factors, extracellular matrix molecules, proteins, enzymes, enzyme inhibitors, receptors, cells, polynucleic acids, lipid-based molecules, polysaccharides and drugs.

37. The apparatus of claim 19, wherein said swellable material further comprises a geometry for directing expansion.
